(19) United States
(12) Patent Application Publication
(10) Pub. No.: US 2022/0296519 A1
(43) Pub. Date: Sep. 22, 2022

(54) PROTEIN STABILIZED LIPOSOMES (PSL) AND METHODS OF MAKING THEREOF

(71) Applicant: Lipotope, LLC, Fairfield, CA (US)

(72) Inventor: Michael ODA, Fairfield, CA (US)

(21) Appl. No.: 17/646,581

(22) Filed: Dec. 30, 2021

Related U.S. Application Data
(60) Provisional application No. 63/132,129, filed on Dec. 30, 2020.

(51) Int. Cl. A61K 9/127

(52) U.S. Cl. CPC A61K 9/1275; A61K 9/1277

(57) ABSTRACT

Compositions and methods for synthesis of protein stabilized liposomes (PSLs) is described. In one aspect, a protein excipient that stabilizes liposome integrity, when incorporated into liposome formulations, is provided. The manufacturing process for production of metastable liposome particles with a half-life of several months is also described. In some aspects the liposome particles may contain a bioactive agent. In some cases, the bioactive agent is a protein, nucleic acid, lipid, or small molecule.

### PRIORITY

This application claims the benefit of U.S. Provisional Patent Application No. 63/132,129, filed on Dec. 30, 2020, and entitled “PROTEIN STABILIZED LIPOSOME (PSL) AND METHODS OF MAKING THEREOF” which application is incorporated herein by reference.

### TECHNICAL FIELD

The present disclosure relates to liposomes, and more particularly, to the stabilization of liposomes for improved delivery of compounds for medical and cosmetic applications.

### BACKGROUND

Liposomes constitute a staple class of nanoparticles that have been used for the delivery of compounds in a wide array of medical and cosmetic applications. Liposomes are phospholipid vesicles, composed mainly of lipids and other hydrophobic elements. Traditionally the lipids are bilayer-forming lipids, cholesterol/sterols, polar lipids, and neutral lipids, wherein polar lipids are predominantly phospholipids. During the synthesis process, bilayer forming lipids form organized bilayer arrays due their propensity to simultaneously present their charged headgroups towards and shield their hydrophobic tail groups away from an aqueous environment. Because spherical structures are the lowest energy state for bilayer lipid arrays, they are the construct that is most often generated. As a result, liposomes are made up of a spherical array of at least one lipid bilayer membrane that may contain an entrapped aqueous internal compartment. In some embodiments, liposomes may not contain an aqueous core or a minimal core due to a high neutral lipid content, multi-laminate layers, or particle size restriction. Water soluble compounds/cargoes incorporated into the liposome synthesis processes are entrapped within the liposome aqueous core (if present), between lipid bilayers of multi-laminate particles, electrostatically associated with the bilayer charged surface elements or chemically tethered to lipids or other elements of the PSL. Hydrophobic compounds incorporated into liposome synthesis processes are incorporated/intercalated into the hydrophobic mileu of the liposome lipid bilayer or chemically tethered to lipids or other elements of the PSL.

Liposomes are particularly useful for the delivery of drugs or other bioactive agents and have been employed in a wide array of therapeutic applications. In particular, they have been used for the systemic delivery of drugs or bioactive agents and when derivatized with ligands (receptor binding moieties or single chain antibodies) they can be directed to target organs and cells. Liposomal formulations of bioactive agents are often superior to drugs injected in free form in that they have a longer circulating half life and achieve higher circulating concentrations with reduced adverse effects. For instance, when used in the delivery of cancer drugs, liposomes have improved the therapeutic index of these drugs by minimizing accumulation in or passage through vulnerable tissues (e.g., the kidneys, and liver) and also by reducing or eliminating the common side effects of nausea, fatigue, and hair loss. Liposomal formulations of the anticancer agent Vincristine exhibit greater efficacy against L1210 leukemia cells than does free Vincristine and have reduced collateral toxicity and adverse effects. Liposomes have also been used for the systemic delivery of protein in the form of vaccines, enzymes, or hormones such as insulin. They have also been used experimentally to carry nucleic acids in the form of synthetic genes, mRNA, cDNA, or guide oligonucleotides to replace defective, disease-causing gene variants, using normal recombinant or CRISPR DNA technology.

Despite their near ubiquitous use, liposomes bear three primary drawbacks: 1) inherent instability; 2) inconsistent manufacturing processes; and 3) methods to stabilize liposomes reduce bioavailability of liposome content. Liposome instability leads to rapid clearance from the body or accumulation in unintended localities in the body.

To address liposome instability, several strategies have been employed. These approaches usually entail the use of lipid derivatives that, while they impart stabilizing characteristics, can also undermine bioavailability and safety. Methods to stabilize liposomes have employed conjugated lipids. A widely utilized approach is incorporation of polyethylene glycol (PEG) or derivatives thereof. A negative consequence of PEG conjugated lipids is reduced liposome bioavailability, resulting in elevated dosing regimens. A more recent strategy to enhance liposome stability incorporates the protein albumin. Albumin's natural role in the body is to convey hydrophobic compounds in the circulation and interstitial fluids. While albumin or conjugated lipid incorporation improve liposome particle stability, it does not substantially improve manufacturing consistency or bioavailability.

### SUMMARY OF THE INVENTION

The current invention addresses liposome stability and consistency of manufacture without compromising bioavailability or safety through the incorporation of apolipoproteins and/or their mimetics (FIG. 1). In their native role, apolipoproteins compose the protein portion of lipoproteins and define many of their biological properties. The apolipoproteins of interest are predominantly associated with high density lipoproteins and intermediate density lipoproteins. In the preferred embodiment, the apolipoprotein is exchangeable. Several exchangeable apolipoproteins have been employed in lipid nanoparticles such as NanoDisks and spherical HDL mimetics, with these particles having a size range from about 7-35 nm in diameter. In contrast, the current invention particle size ranges from about 60 nm to over about 3.5 μm in diameter. The presence of exchangeable apolipoproteins on particles of this size is highly unanticipated, as studies of apolipoproteins for over the past 60 years have consistently described these proteins as associated with particles of less than 35 nm in diameter. This has led the scientific community to conclude that these molecules do not stably associate with or catalyze the formation of particles of the size range described in this invention (about 60 nm to over about 3.5 μm in diameter).

A benefit of particle size is that larger size facilitates different modes of translocation into the body not available to smaller particles and particle size influences particle stability. For instance, sinus transport into trigeminal nerves is optimally mediated by particles of about greater than 1 μm in diameter. Additionally, larger particles are more inclined to disassemble after entry into the body, resulting in more localized cargo delivery and thus larger particles are preferable for the administration of compounds that yield systemic adverse effects like damage the liver and kidney. Specifically, the invention is directed to a metastable assembly of apolipoproteins, lipid component, and bioactive agent incorporated as protein stabilized liposome (PSL) particles with a diameter from about 60 nm to over about 3.5 μm and with a half-life of 2 years, or at least about three months, or preferably at least about six months, or most preferably at least about 3 years when the PSLs are stored at 4° C. The process by which protein stabilized liposome particles are manufactured is highly reproducible and does not compromise the potency/bioactivity of associated cargoes, with a batch to batch consistency of over 80%.

The present invention describes a composition and method of production for protein stabilized liposomes PSL, which are highly stable and bear functional characteristics common to classical liposomes. The method of synthesis yields consistent results, with a greater than 80% batch to batch success rate (FIG. 2). The element of manufacture that confers stability to PSLs is the presence of one or more apolipoprotein. In the preferred embodiment, the apolipoprotein is exchangeable. In this invention, exchangeable apolipoproteins guide the formation/coalescing of PSL components into large metastable particles. PSLs have utility in the delivery of one or more bioactive agents that include proteins, nucleic acids, lipids, chemicals, and small molecules.

The invention provides compositions and methods for delivery of a bioactive agent to an individual. Methods of administration include techniques generally applied to liposomes, consisting of aerosol, instillation, insufflation, intraperitoneal, intrathecal, intravenous, ocular, parenteral, sinus, transdermal, transmucosal, transpulmonary, transserous, and a combination thereof.

### Properties of PSL

Size—at least >50 nm in diameter, wherein major populations exist at approximately 220 nm and 2.2 μm in diameter. The shape of PSLs is spherical and ellipsoidal. Some PSL embodiments contain an aqueous core. Other PSL embodiments are solid multi-lamellar particles. The size, composition, and multi-laminate nature of the PSL produced is governed by the lipid composition of the particle, the nature of the cargo(es) and the ratio of lipids and cargo present.

Stability—In the preferred embodiment PSLs maintain particle integrity/size distribution for at least 1 year when stored at 4° C. and for at least 3 months when stored at room temperature (20-22° C.). In the preferred embodiment the concentration of apolipoprotein in a solution is about 5 μg/mL. When apolipoprotein concentration is below about 500 ng/mL, PSL exhibits reduced stability. When the concentration of apolipoprotein approaches above 11 mg/mL, the apolipoprotein becomes unstructured. The acceptable range for the initial concentration of apolipoprotein in a solution per the manufacturing process for PSL stability is about between 100 ng/mL to 11 mg/mL by weight. The acceptable range for the final concentration of apolipoprotein with respect to the volume of PSL for PSL stability is between about 100 ng/mL (0.1 μg/mL) to about 11 mg/mL by weight. In an embodiment, the final concentration of the apolipoprotein in a solution is between about 0.5 μg/mL to about 200 μg/mL. The apolipoprotein concentration dependence of PSL stability enables programmed disassembly upon entry into target tissue. For instance, PSL entry into the body significantly reduces the concentration of the apolipoprotein element and if PSLs are administered with an apolipoprotein above 500 ng/ml (the initial concentration of apolipoprotein used in the manufacturing) and upon entry is diluted to below 100 ng/ml (or below 0.1 ng/mL of the final concentration of apolipoprotein), PSLs will disassemble near the site of application. Further, while PSL are stable at room temperatures, at 65° C. PSLs disassemble, this is predominantly due to the thermal stability of apolipoproteins, which lose structural integrity at above 65° C. As with liposomes, agitation and shear force are a major contributing factor to spontaneous PSL particle disassembly but PSL are sufficiently stable to withstand the agitation and shear forces of standard laboratory and manufacturing processes.

Function—PSLs, like traditional liposomes, are able to transport bioactive compounds to the body. Unlike bioactive agents on traditional liposomes, bioactive agents incorporated into PSLs retain or bear enhanced bioavailability.

Manufacturing Process—The PSL production process entails methodologies that yield liposomes in the absence of significant shear force or cavitation. In a preferred methodology, the approach includes microfluidic lamellar flow mixing, Dean vortex mixing, and micro turbulent flow mixing. These microfluidic processes gently mix aqueous and lipid phases for the stable formation of delicate liposome and lipid nanoparticle constructs. The gentle nature of these processes allows PSLs to form and coalesce into metastable lipid particles. The preferred methodology employs a two channel microfluidic flow cell, wherein one channel contains apolipoprotein in saline or other aqueous buffer and the other channel contains bioactive cargo plus liposome lipid component (e.g., palmitoyl oleoyl phosphatidylcholine (POPC), dimyristoylphosphatidylcholine (DMPC), bilayer forming lipids, non-bilayer forming lipids, polyethylene glycol (PEG) conjugated lipids and other lipids commonly employed in liposome synthesis) in ethanol or other lipid solvating solvent that is miscible with an aqueous solution (FIG. 3). In a preferred embodiment, the bioactive cargo includes one or more bioactive agents. This method of synthesis yields consistent results, with a greater than 80% batch to batch success rate (FIG. 2). The apolipoprotein ingredient is the critical element that confers enhanced consistency of manufacture. Exchangeable apolipoproteins guide the formation/coalescing of PSL components into large metastable particles, thereby avoiding the formation of unstable constructs that compromise yield and consistency of particle size distribution, as commonly observed with traditional liposome synthesis methodology. This is demonstrated by the observation that the stability of PSL increases with increasing apolipoprotein content. A preferred methodology employs a ratio of apolipoprotein (or mimetic) to cargo/lipid of 1:150 w/w. PSLs become unstable when the ratio approaches about 1:200 w/w for apolipoprotein (or mimetic) to cargo/lipid. Batch success is defined by a combination of particle population distribution and stability. Unsuccessful batches are generally unstable and disassemble within minutes of synthesis. Batch success is indicated when a formulation batch is stable at room temperature for more than 2 days (FIG. 5). Instability in this case is indicated by the separation of bioactive agent cargo from PSL and the appearance of a two-phase solution or lipid droplets (“oiling off”) on the container walls or solution surface through droplet coalescence that would account for at least 30% of the bioactive agent or formation of a non-suspendable sediment. Further, for each formulation of PSL with bioactive agent a profile of optimal particle size distribution will be created (FIG. 6). Batch success is confirmed when the profile of the newly synthesized stable PSLs bears at least an 80% identity with the appropriate predefined particle distribution profile (FIG. 2).

### Definitions

The terms “approximately” or “about in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The terms “peptide” or “polypeptide” as used herein generally refers to a polymer of at least two amino acids linked to one another by peptide bonds. Peptides may include moieties other than amino acids (e.g., glycoproteins) and/or may be otherwise processed or modified. Those of ordinary skill in the art will appreciate that a “peptide’ or “polypeptide” can be a complete polypeptide chain as produced by a cell or can be a functional portion thereof. In some embodiments, the term “protein’ refers to a single polypeptide chain. In some embodiments, a protein may comprise more than one polypeptide chain, for example linked by one or more disulfide bonds or associated by other means.

The term a “small molecule” as used herein is understood in the art to be an organic molecule that is less than about 2000 g/mol in size. In some embodiments, the small molecule is less than about 1500 g/mol or less than about 1000 g/mol. In some embodiments, the small molecule is less than about 800 g/mol or less than about 500 g/mol. In some embodiments, small molecules are non-polymeric and/or non-oligomeric. In some embodiments, small molecules are not proteins, peptides, or amino acids. In some embodiments, small molecules are not nucleic acids or nucleotides. In some embodiments, small molecules are not saccharides or polysaccharides.

The term “nanoparticle” as used herein refers to any entity having a diameter of less than 500 microns (μm). Typically, particles have a greatest dimension (e.g., diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit but are small enough to avoid accumulation in the liver. Nanoparticles having a spherical shape are generally referred to as “nanospheres.”

The term “hydrophilic” as used herein refers to substances that have strongly polar groups that readily interact with water.

The term “lipophilic” as used herein refers to compounds having an affinity for lipids.

The term “amphiphilic” as used herein refers to a molecule combining hydrophilic and lipophilic (hydrophobic) properties.

The term “hydrophobic” as used herein refers to substances that lack an affinity for water; tending to repel and not absorb water as well as not dissolve in or mix with water.

The term “amphipathic alpha helix” as used herein is an often-encountered secondary structural motif in biologically active peptides and proteins. Amphipathicity corresponds to the segregation of hydrophobic and polar residues between the two opposite faces of the α-helix, a distribution well suited for membrane binding. An amphipathic α-helix is defined as an alpha helix with opposing polar and nonpolar faces oriented along the long axis of the helix. An amphipathic α-helix is a structure/function motif involved in lipid interaction; this observation was first observed apolipoproteins. Amphipathic alpha helices have since been described in other putative lipid-associating proteins, including certain polypeptide hormones, polypeptide venoms, polypeptide antibiotics, complex transmembrane proteins, and the human immunodeficiency virus glycoprotein. In addition, amphipathic helixes involved in both intra- and intermolecular protein-protein interactions have been described in a number of proteins, including globular proteins, calmodulin-regulated protein kinases, and coiled-coil-containing proteins (e.g., kinesin (2-helix coiled-coil) collagen (3-helix coiled-coil)).

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including, for example: attached to a monomer of such polymer (by covalent, ionic, or other binding interaction), physical admixture, enveloping the agent in a coating layer of polymer, incorporated into the polymer, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

The terms “subject” or “patient” or “individual” as used herein refers to any organism to which particles produced by a microfluidic system as described herein may be administered, e.g., for experimental, therapeutic, diagnostic, and/or prophylactic purposes. Typical subjects include animals (e.g., mammals such as mice, rats, rabbits, non-human primates, and humans) and/or plants. A subject or a patient as used herein includes a human, a mammal, or generally an animal.

As used herein, “subject, patient or individual” refers to any prokaryote or eukaryote to which one desires to deliver a bioactive agent. In some embodiments, the individual is a prokaryote such as a bacterium. In other embodiments, the individual is a eukaryote, such as a fungus, a plant, an invertebrate animal, such as an insect, or a vertebrate animal. In some embodiments, the individual is a vertebrate, such as a human, a nonhuman primate; an experimental animal, such as a mouse or rat; a pet animal, such as a cat or dog; or a farm animal, such as a horse, sheep, cow, or pig; or an avian animal, such as a chicken, turkey or duck; or a reptilian animal, a such as a lizard or snake.

The term “protein stability” as used herein refers to at least about 80% of the protein structure retaining functionality or secondary structure composition over a set period.

The term “miscibility” as used herein refers to the ability of a liquid solute to dissolve in another liquid as a solvent. We can define miscible liquids as liquids that can mix to form a homogeneous solution.

The term “apolipoproteins” as used herein refers to apolipoproteins, their natural occurring variants, truncation variants, single and multiple amino acid substitution variants, peptide mimetics, and chimeric variants thereof. Apolipoproteins are the primary protein constituent of lipoproteins that naturally transport lipids in blood and lymphatic circulation and are central to lipid metabolism, cardiovascular health and disease. In this invention apolipoproteins impart stability to PSLs and consistency to their manufacture. Apolipoproteins are bound to lipoproteins and PSLs through a combination of hydrophobic bonding between the hydrophobic face of the apolipoprotein amphipathic alpha helices and the hydrophobic tail groups of lipoproteins and PSL lipids, and electrostatic bonding between the charged residues of the apolipoprotein and the polar head groups of lipoproteins and PSL lipids. Examples of apolipoproteins that may be used for the formation of PSLs include but not limited to: apolipoprotein A-I (apoA-I), apolipoprotein A-II (apoA-II), apolipoprotein A-IV (apoA-IV), apolipoprotein A-V (apoA-V), apolipoprotein C-I (apoC-I), apolipoprotein C-II (apoC-II), apolipoprotein C-III (apoC-III), apolipoprotein D (apoD), apolipoprotein E (apoE), apolipoprotein H (apoH), apolipoprotein J (apoJ), apolipoprotein M (apoM), or fragments, natural variations, isoforms, amino acid substitution variants, analogs or chimeric forms thereof. In the preferred embodiment the apolipoprotein is an exchangeable apolipoprotein.

The term “exchangeable apolipoproteins” as used herein refers to apolipoproteins capable of exchanging between lipoprotein particles and can transiently dissociate from the lipoprotein surface in a lipid-free form via spontaneous dissociation or displacement by another exchangeable apolipoprotein (e.g., apoA-I, apoA-II, apoA-IV, apoC-I, apoC-II, apoC-III, apoD, apoE, apoJ, and apoM), as opposed to the non-exchangeable apolipoproteins that remain with one lipoprotein particle from biosynthesis to catabolism (e.g., apolipoproteins B-48 and B-100). A distinguishing feature of exchangeable apolipoproteins is the presence of internal 11-residue-long amino acid repeats. In apoA-I, apoA-IV, and apoE the 11-mer repeats have evolved into multiple 22-mer tandem repeats. Most of these 22-mer repeat units form amphipathic alpha helices. Many of the 22-mer repeats have proline residues at the first, and only at the first, position. These 22-mer repeats are predominantly connected by short 2-4 residue loop segments that impart a degree of flexibility to exchangeable apolipoproteins that is not present in non-exchangeable apolipoproteins.

The terms “mimetics” or “peptide mimetics” as used herein refers to molecules that bear one or more amphipathic alpha helix and can substitute for apolipoproteins due to their lipid binding and discoidal lipoprotein particle-forming activity (in the absence of neutral lipid). In the preferred embodiment of a mimetic, the mimetic peptide reproduces apoA-I's ability to form high density lipoprotein-like lipoproteins. In general, mimetic peptides need not have sequence homology to apoA-I, the primary physical property requirement is the ability to form a class A amphipathic helix, which is a structural property necessary to reproduce apoA-I's ability to form small discrete discoidal particles via mixing with bilayer-forming lipids through cavitation, emulsification, or sonication. Mimetic peptide lipid binding properties can be improved by blocking the end groups of these peptides with an acetyl group and an amide group. Candidate mimetics may bear additional apolipoprotein traits, for instance a prototypic apolipoprotein mimetic named “4F” produces lipoprotein particles with cardioprotective properties similar to high density lipoproteins, the primary lipoprotein to which apoA-I is naturally associated. Importantly, the peptide 4F form small discrete discoidal particles via mixing with bilayer-forming lipids through cavitation, emulsification, or sonication. In some embodiments, the apolipoprotein may be modified to bear a non-native cysteine residue to form a chemical link through either disulfide or maleimide chemistry to a bioactive agent or another apolipoprotein. Furthermore, this cysteine residue may be used to link multiple apolipoproteins to modulate the exchangeability of the apolipoprotein, wherein these linkages limit protein structure dynamics and thereby limit the ability of the apolipoprotein to dissociate from lipoproteins. Chimeric apolipoprotein molecules are also provided and may be used to incorporate various additional functional properties into the PSLs of the invention.

The term “chimeric” as used herein refers to two or more molecules that are capable of existing separately and are joined to form a single molecule having the desired functionality of all of its constituent molecules. The constituent molecules of a chimeric molecule may be joined synthetically by chemical conjugation or, where the constituent molecules are all polypeptides or analogs thereof, polynucleotides encoding the polypeptides may be fused together recombinantly, such that a single continuous polypeptide is expressed. Such a chimeric molecule is termed a fusion protein. A “fusion protein” is a chimeric molecule in which the constituent molecules are entirely composed of polypeptides and are attached (fused) to each other such that the chimeric molecule forms a continuous single polypeptide. The various constituents can be directly attached to each other or can be coupled through one or more linkers. In this invention the term “chimeric” applies to the apolipoprotein component of PSLs.

The term “chimeric apolipoprotein” as used herein refers to the apolipoprotein component of PSLs and variants or mimetics thereof, which are amended in capability or characteristics by chemically bound functional, biologically active, or property enhancing/altering elements. Chimeric augmentation of apolipoproteins may also affect the behavior or properties of the PSL cargo, for instance, one or more targeting moieties (e.g., variable domain of a single chain antibody) and/or an element with biological activity that may amplify or synergistically enhance the activity or features of PSL cargo. In one embodiment, a chimeric apolipoprotein includes a targeting moiety that is not intrinsic to the native apolipoprotein (e.g., the variable domain of a single chain antibody, S. cerevisiae C. mating factor peptide, folic acid, glycans, antigenic portions of an infective agent, transferrin, lactoferrin, or extracellular portion of a receptor). In another embodiment, a chimeric apolipoprotein includes a moiety with a desired biological activity that augments and/or synergizes with the activity of a bioactive agent incorporated into the PSL (e.g., bactenecin, colicin, cyclosporine, defensin, echinocandin, hepcidin, histatin-5, magainin peptide, melittin, or N-terminal lactoferrin peptide) or an enzyme (e.g., proteases, lipases, or amylases). In another embodiment, a chimeric apolipoprotein includes a moiety that alters the luminescent or spectroscopic behavior of the apolipoprotein, (e.g., a fluorophore or chromophore, which may be a small molecule chromophore like indigo or a multichromatic reporter small molecule like phenolphthalein, or fluorophore small molecule like rhodamine or quantum dot, or a fluorescent protein like green fluorescent protein or a combination thereof to modulate light response or reporter behavior). In another embodiment, a chimeric apolipoprotein may include a functional element that has hormone signaling properties and may be a peptide hormone (e.g., amylin, angiotensin, calcitonin, gastrin, ghrelin, glucagon, growth hormone, follicle-stimulating hormone, insulin, leptin, oxytocin, prolactin, renin, somatostatin, thyroid-stimulating hormone, vasopressin or vasoactive intestinal peptide), or may be a small molecule lipid-derived hormone like a steroid (e.g., cortisol, estradiol, or testosterone), or may be a small molecule acid-derived hormone (e.g., melatonin or tryptophan). In another embodiment, a chimeric apolipoprotein may include a functional moiety intrinsic to another apolipoprotein (e.g., an apolipoprotein targeting moiety formed approximately by amino acids 130-150 of human apoE, which comprises the receptor binding region recognized by members of the low-density lipoprotein receptor family). In other embodiments, a functional moiety may be added chemically or recombinantly to produce a chimeric apolipoprotein.

The term “linker’ or “spacer” as used herein in reference to a chimeric molecule refers to any molecule that links or joins the constituent molecules of the chimeric molecule. A number of linker molecules are commercially available, for example from Pierce Chemical Company, Rockford Ill. Suitable linkers are well known to those of skill in the art and include but are not limited to straight or branched-chain carbon linkers, heterocyclic carbon linkers, or peptide linkers. Where the chimeric molecule is a fusion protein, the linker may be a peptide that joins the proteins comprising a fusion protein. Although a spacer generally has no specific biological activity other than to join the proteins or to preserve some minimum distance or other spatial relationship between them, the constituent amino acids of a peptide spacer may be selected to influence some property of the molecule such as the folding, net charge, or hydrophobicity. In some embodiments, a chimeric lipid binding polypeptide. Such as a chimeric apolipoprotein, is prepared by chemically conjugating the apolipoprotein molecule and the functional moiety to be attached. Means of chemically conjugating molecules are well known to those of skill in the art. Such means will vary according to the structure of the moiety to be attached, but will be readily ascertainable to those of skill in the art.

The term “bioactive agent” as used herein refers to any compound or composition having biological, including therapeutic or diagnostic, activity. A bioactive agent may be a pharmaceutical agent, drug, compound, or composition that is useful in medical treatment, diagnosis, or prophylaxis. A bioactive agent may be a pharmaceutical agent, cosmeceutical, nutraceutical, drug, nucleic acid, peptide, lipid, compound, or composition that is useful in medical treatment, diagnosis, or prophylaxis. The bioactive agent may be a two-(or more) component system wherein multiple elements are required to interact to yield biological activity or the bioactive agent augments the activity of another bioactive agent or the biological activity of elements inherent to the subject, patient or individual.

The term “cargo” as used herein refers to molecules or agents that have integrated in PSLs: hydrophobically bound to the acyl chains of and intercalated into the lipid bilayer leaflet(s), electrostatically bound to the PSL surface, encapsulated within the PSL aqueous core, sandwiched between PSL bilayers (for multilaminate PSLs) or chemically linked to the apolipoprotein component. Bioactive agent cargo incorporated into PSL as described herein generally include at least one hydrophobic (e.g., lipophilic) region capable of associating with or integrating into the hydrophobic portion of a lipid bilayer or a charged domain that electrostatically binds to the PSL lipid surface or is chemically bound to the apolipoprotein or lipid component of the PSL. In some embodiments, at least a portion of the bioactive agent is intercalated between lipid molecules in the interior of the PSL. In some embodiments, at least a portion of the bioactive agent is electrostatically bound to charged elements on the surface of or between leaflets of multilaminate PSL. In some embodiments, at least a portion of the bioactive agent is encapsulated within the aqueous core of the PSL. In some embodiments, the bioactive agent is chemically bound/linked to the apolipoprotein or lipid component of the PSL. Methods to incorporate bioactive agents into PSL include but are not limited to the following: 1) the bioactive agent intercalates into the lipid milieu of the PSL through hydrophobic bonding of a naturally occurring hydrophobic element of the bioactive agent, 2) the bioactive agent intercalates into the lipid milieu of the PSL through hydrophobic bonding of a hydrophobic element that is chemically linked to the bioactive agent, 3) the bioactive agent is incorporated into an aqueous core of PSL, in embodiments wherein PSL bears an aqueous core, 4) the bioactive agent is attached to the lipoprotein through a chemical linkage or in the case of a bioactive protein/peptide, the sequence is amended to or incorporated into the apolipoprotein amino acid sequence through recombinant DNA technology or solid state chemistry, 5) charge moieties on the bioactive agent binds it to charged elements on the lipid or apolipoprotein through electrostatic interaction, or 6) the bioactive agent binds to another bioactive agent that has been otherwise incorporated into the PSL particle (FIG. 1). Examples of bioactive agents that may be incorporated into delivery particles in accordance with the invention include, but are not limited to, anesthetics, antibiotic or antimicrobial agents (e.g., antibacterial, antifungal, and antiviral), anticancer agents, antimetabolic agents, antineoplastic agents, antigen (e.g., vaccine), bioactive lipids (such as, for example cannabinoids and steroids), chromophores, fluorescent compounds, herbicides, insecticides, lipids, neurotransmitters, nutrients, peptides, pesticides, photosensitizing agent (as used in photodynamic therapy), proteins (such as, for example, cell receptor proteins, enzymes, hormones, and single chain antibodies), quenching agents, radiolabels (such as, for example radioactive and non-radioactive isotopes and radioisotope-labeled compounds and proteins), or paramagnetic compounds. In other embodiments, the bioactive agent is predominantly hydrophobic are known in the art and include, all-trans retinoic acid, annamycin, camptothecin, cannabinoids, cannabis extract, docetaxel, doxorubicin, etiopurpurins, nystatin, paclitaxel and tetrahydrocannabinol. Bioactive agents that include at least one hydrophobic region are known in the art and include, but are not limited to α-tocopherol, cyclosporin, cortisone, diazepam, etoposide, griseofulvin, ibuprofen, lipopolysaccharides, proleukin, taxane, vitamin A, and vitamin E. In some embodiments, a bioactive agent incorporated into a PSL of the invention is a non-polypeptide. In some embodiments, for administration to an individual, a bioactive agent and the PSL that includes the bioactive agent are substantially nonimmunogenic when administered to an individual. In some embodiments, a bioactive agent is a small molecule, nucleic acid, or a biologic.

The term “controlled release” as used herein refers to release of a bioactive agent from a formulation at a rate that the blood concentration of the agent in an individual is maintained within the therapeutic range for an extended duration, over a time period on the order of hours, days, weeks, or longer. PSL may be formulated in a bioerodible or nonbioerodible controlled matrix, a number of which are well known in the art. A controlled release matrix may include a synthetic polymer or copolymer, for example in the form of a hydrogel. Examples of such polymers include polyesters, polyorthoesters, polyanhydrides, polysaccharides, poly(phosphoesters), polyamides, polyurethanes, poly(imidocarbonates) and poly(phosphaZenes), and poly-lactide-co-gly collide (PLGA), a copolymer of poly(lactic acid) and poly(glycolic acid). Collagen and fibrinogen containing materials may also be used.

The term “batch success” is defined as the certification of a batch as having met formulation-specific criteria. These criteria are a combination of a demonstration of short-term stability and the batch particle population distribution profile (FIGS. 2 and 5). Unsuccessful batches are generally unstable and disassemble within minutes of synthesis. Batch success is indicated when a formulation batch is stable at room temperature for more than 48 hours. Instability in this case is indicated by the separation of bioactive agent cargo from PSL and the appearance of a two-phase solution or lipid droplets (“oiling off”) on the container walls or at the solution surface through droplet coalescence that would account for at least 30% of the bioactive agent or formation of a non-suspendable sediment (FIG. 5). Further, for each formulation of PSL, with bioactive agent, a profile of optimal particle size distribution is generated. Batch success is confirmed when the particle size distribution profile of the newly synthesized 48-hour stable PSLs bears at least an 80% identity with the appropriate PSL—bioactive agent specific optimal particle distribution profile. Particle size distribution profiles are determined by native polyacrylamide gel electrophoresis or dynamic light scatter methodologies (FIG. 6).

The term “lipid component” as used herein refers to a lipid element of PSL that can be either a bilayer-forming or non-bilayer-forming lipid. A “bilayer-forming lipid” refers to a lipid that is capable of forming a lipid bilayer with a hydrophobic interior and hydrophilic exterior. Any bilayer-forming lipid that is capable of associating with apolipoproteins or mimetics may be used in accordance with this invention. Bilayer-forming lipids include but are not limited to alkylphospholipids, ether lipids, glycolipids, phospholipids, plasmalogens, and sphingolipids. In some embodiments the lipid-bilayer includes phospholipids. Examples of suitable phospholipids includes but not limited to: dipalmitoyl phosphatidylcholine (DMPC), dimyristoyl phosphoglycerol (DMPG), palmitoyl oleoyl phosphatidylcholine (POPC), dipalmitoyl phosphatidylcholine (DPPC), dipalmitoyl phosphatidylserine (DPPS), dipalmitoyl phosphatidylglycerol (DPPG), distearoyl phosphatidylglycerol (DSPG), egg yolk phosphatidylcholine (egg PC), soy bean phosphatidyl choline, phosphatidylinositol, phosphatidic acid, sphingomyelin, and cationic phospholipids. Examples of other suitable bilayer-forming lipids include cationic lipids and glycolipids.

Some embodiments of PSL contain non-bilayer-forming lipids. Such lipids include, but are not limited to, alkylphospholipids, cardiolipin, cationic lipids, ceramide, cerebrosides, cholesterol, diacylglycerol, ergosterol, etherlipids, gangliosides, lysophospholipids, monoacylglycerol, oxysterols, phosphatidyl ethanolamine (this lipid may form bilayers under certain circumstances), plant sterols, plasmalogens, prostaglandins, sitosterol, sphingosine, and triacylglycerol. In some embodiments, a lipid used for preparation of a PSL may include one or more bound functional moieties, such as targeting moieties, bioactive agents, or tags for purification or detection.

The term “moiety” as used herein refers to a substituent of a molecule. A moiety generally describes a larger substituent of a molecule that is not used to describe generally a smaller functional group. As used in the current application, a moiety can comprise a functional group. Moreover, in the current instance, moiety and functional group could also be synonymous. The term “functional group” as used herein refers to a specific part or moiety within a molecule that may be responsible for a particular chemical reaction of the molecule. The same functional group or moiety will undergo the same or similar chemical reaction regardless of the size of the molecule. As for the term “modifying moiety” as used herein, it refers to a part of a molecule wherein the part performs a chemical reaction on a substrate that modifies, transforms, or alters the substrate. Further, the “modifying moiety” may augment or add a functional trait to the molecule with which it is attached, as for example enhanced affinity to an antigen, lectin, ligand, or receptor; enhanced immunogenicity as by a hapten; altered spectral or fluorescent properties; additional chemical reactivity as by primary amines or sulfhydryl groups; or enhancement of enzymatic activity as by the addition of a charged group that draws substrate into the active site.

### BRIEF DESCRIPTION OF FIGURES

FIG. 1 is an illustration of PSLs, classes of cargoes, modifications and theorized association of stabilizing apolipoprotein.

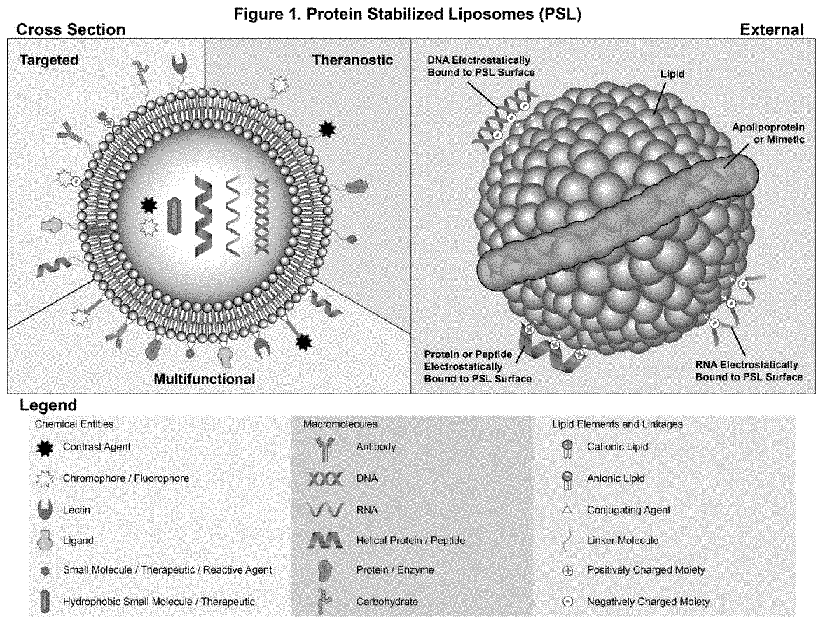

FIG. 2 shows the consistency of PSL particle size distribution as determined by dynamic light scatter. The PSL is composed of a ratio of 1350:150:1 of CBD distillate:DMPC:apolipoprotein (% weight). Particles were synthesized using staggered herringbone microfluidic mixer as illustrated in FIG. 3 and particle size quantified by dynamic light scatter. Measures are the aggregate of 5 separate measurements.

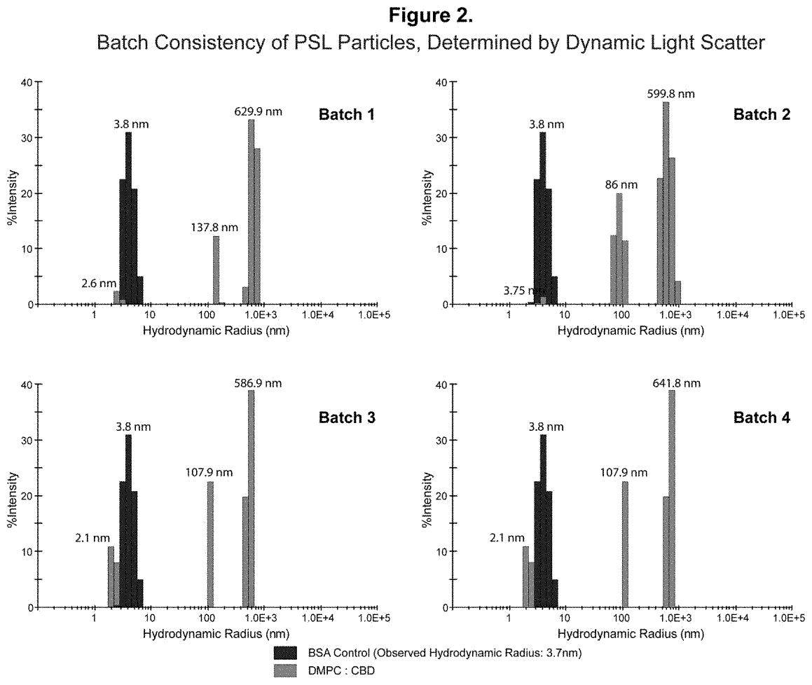

FIG. 3 is an illustration of the preferred method of PSL production. In this instance a staggered herringbone microfluidic mixer is employed to combine two flow channels but other microfluidic mixing geometries and methodologies may be utilized.

FIG. 4 shows the multi-year stability of various PSL formulations is demonstrated by dynamic light scatter. Multiple preparations of PSLs were stored at 4° C. for 1, 2, and 3 years and compared to freshly prepared formulations. Particle size of the primary population was maintained within 10% of the value of freshly prepared PSLs. Particle size was quantified by dynamic light scatter. Measures are the aggregate of 5 separate measurements.

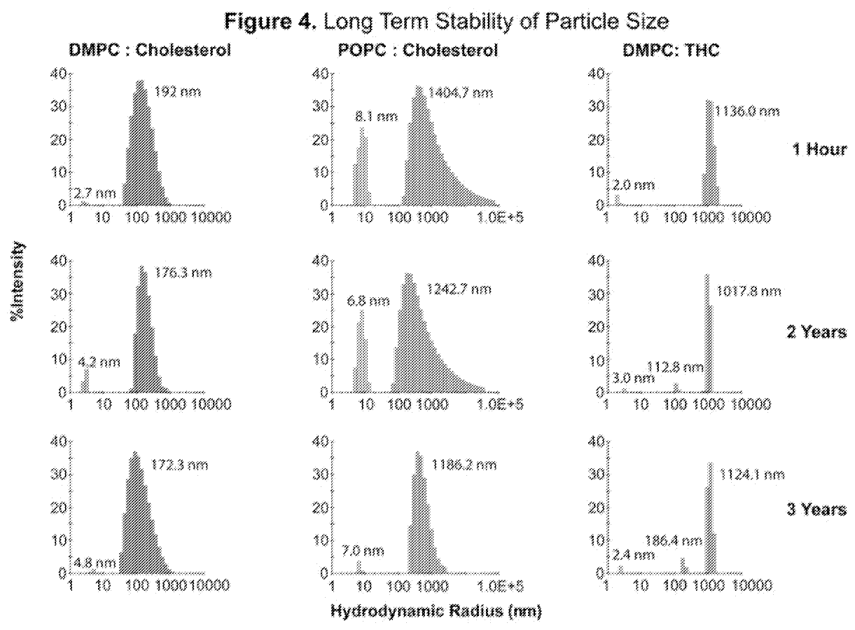

FIG. 5 shows the effect of apolipoprotein on liposome stability is illustrated by the comparison of two preparations of DMPC:THC liposomes, wherein apolipoprotein was excluded and included in a 1350:150:1 mix of THC distillate:DMPC:apolipoprotein (% weight). Samples were stored at room temp (−20° C.). Particle instability is demonstrated by the deposition of THC distillate on the tube wall and formation of a sediment that is not suspendible. In contrast PSLs maintain their suspensibility and exhibit minimal deposition of cargo on tube wall. Maintenance of suspensibility after 48 hours of room temp storage is a criteria of preparation success. Particle size was quantified by dynamic light scatter. Measures are the aggregate of 5 separate measurements.

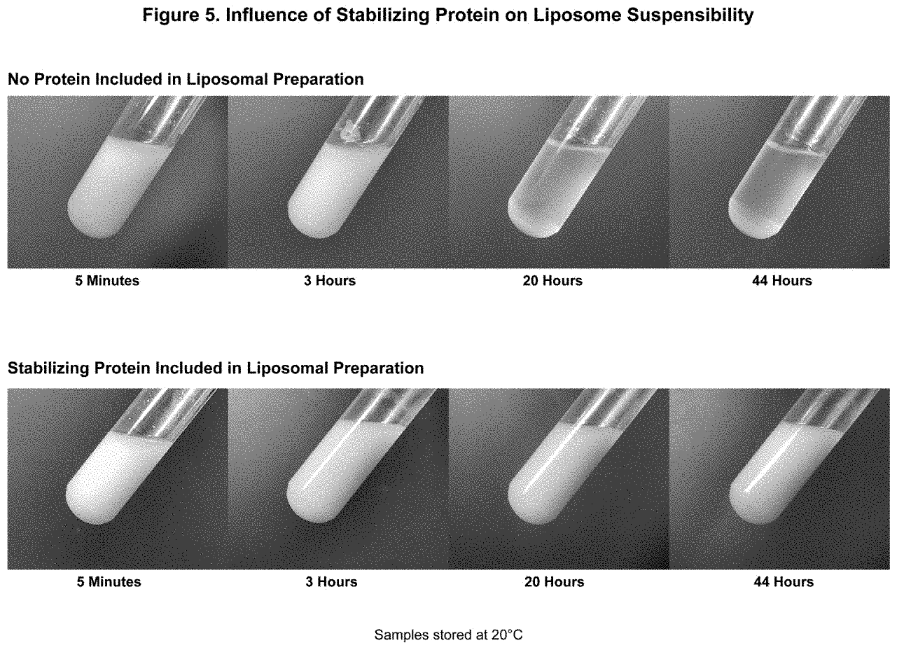

FIG. 6 shows the effect of PSL particle composition on PSL particle size distribution. Four unique compositions were examined: DMPC Cholesterol (1350:150:1 ratio of DMPC:Cholesterol:apolipoprotein (% weight)), POPC Cholesterol (1350:150:1 ratio of DMPC:Cholesterol:apolipoprotein (% weight)), DMPC:THC (1350:150:1 ratio of THC Distillate:DMPC:apolipoprotein (% weight)), DMPC:CBD (1350:150:1 ratio of CBD Distillate:DMPC:apolipoprotein (% weight)). Particles were synthesized using a staggered herringbone microfluidic mixer as illustrated in FIG. 3. Particle size was quantified by dynamic light scatter. Measures are the aggregate of 5 separate measurements.

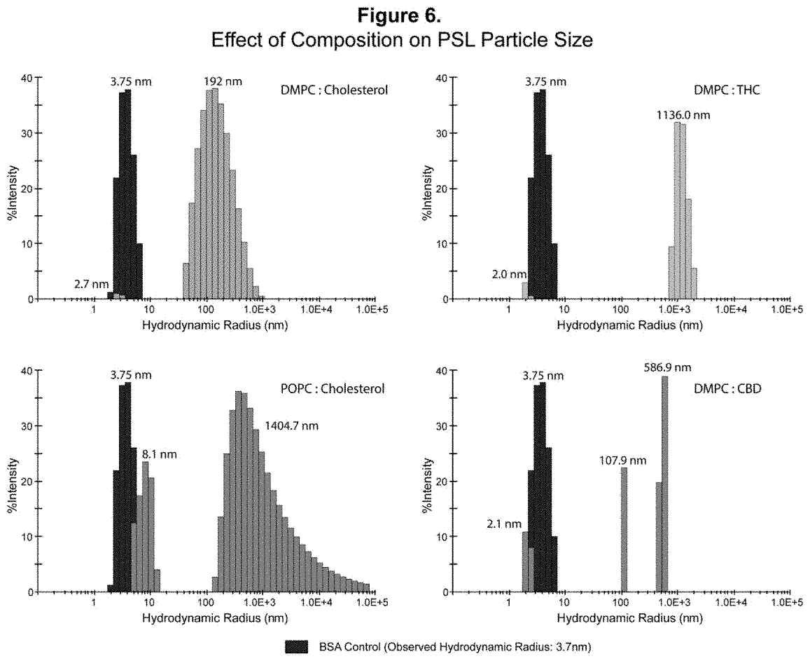

### DETAILED DESCRIPTION

The invention provides compositions and methods for synthesis of a delivery vehicle conveying a bioactive agent(s) to a subject, patient or individual. Delivery vehicles are provided in the form of a bioactive agent(s) incorporated into a protein stabilized liposome (PSL) that includes an apolipoprotein and a lipid bilayer.

In an embodiment, the disclosure is directed to a synthetic PSL that includes an apolipoprotein; a bioactive agent; and a liposome; wherein the synthetic PSL is configured to have a diameter greater than about 60 nm. In a further embodiment, the concentration of the apolipoprotein or the mimetic in the PSL is between about 100 ng/mL to about 11 mg/mL by weight. In an embodiment, the concentration of the apolipoprotein or the mimetic in the PSL is about 5 μg/mL. In an embodiment, the PSL is configured to have a diameter from about 60 nm to about 3.5 μm. In an embodiment, the PSL is configured to have a diameter over about 2.0 μm. In an embodiment, the PSL is configured to be stabilized when stored at about 4° C. for at least about three months. In an embodiment, the PSL is configured to be stabilized when stored at about 4° C. for at least about six months.

In an embodiment, the apolipoprotein comprises an exchangeable apolipoprotein capable of displacing a resident apolipoprotein from a preformed PSL. In an embodiment, the exchangeable apolipoprotein comprises a motif of approximately 20-24 amino acids configured to form an amphipathic alpha helix. In an embodiment, the exchangeable apolipoprotein comprises a plurality of the motif of approximately 20-24 amino acid configured to form the amphipathic alpha helix. In an embodiment, the motif comprises an approximate 22-residue peptide of Glu, Lys, and Leu arranged substantially periodically to form an amphipathic alpha helix with a polar face and a non-polar face. In an embodiment, the exchangeable apolipoprotein comprises a peptide configured to mimic an amphipathic helical domain of the apolipoprotein, wherein a plurality of positively charged residues are configured at the polar-nonpolar face interface and a plurality of negatively charged residues configured at the center of the polar face. Exchangeable apolipoprotein is selected from the group consisting of apoA-I, apoA-II, apoC-I, apoC-II, apoC-Ill, apoD, apoE, apoH, apoJ, apoM, apoA-IV, apoA-V, natural variation, isoform, or single or multi-amino acid substitution thereof.

In an embodiment, the synthetic protein stabilized liposome is among a plurality of protein stabilized liposomes; and wherein the plurality of protein stabilized liposomes has more than 80% batch success rate. In an embodiment, the apolipoprotein is selected from the group consisting of apoA-I, apoA-II, apoC-I, apoC-II, apoC-Ill, apoD, apoE, apoH, apoJ, apoM, apoA-IV, apoA-V, fragment, natural variation, isoform, single or multi-amino acid substitution, analog, chimera, mimetic, or combination thereof. In an embodiment, an amino acid substitution comprises a specific amino acid residue substituted, for instance, by a cysteine residue in the apolipoprotein; and wherein the cysteine is configured to form an intramolecular or an intermolecular disulfide bond. Such bonding modifies the “exchangeability” of the apolipoprotein by modulating its structural flexibility. In an embodiment, the chimeric form comprises a apolipoprotein molecule, the apolipoprotein molecule comprise a functional moiety with a biological activity selected from the group consisting of an anti-microbial activity, anti-cancer activity, anti-viral activity, anti-angiogenesis activity, anti-immunological activity, anti-inflammatory activity, anti-atherosclerosis activity, anti-fungal activity, anti-parasitic activity, anti-thrombotic activity, chromatic activity, enzymatic activity, fluorescent activity, hormone signaling activity, metabolic activity, paramagnetic activity, psychoactive activity, signal quenching activity, radioactivity, receptor binding or targeting activity and a combination thereof. In an embodiment, the functional moiety augment or synergizes the activity of the bioactive agent. In an embodiment, the bioactive agent is selected from the group consisting of a protein, a nucleic acid, a chemical, a small molecule, and a combination thereof. In an embodiment, wherein the mimetic is configured to form substantially a class A amphipathic helix or substantially to mimic the ability of apoA-I to form high density lipoprotein-like lipoproteins. In general, mimetic peptides need not have sequence homology to apoA-I, their primary physical property requirement is the ability to form a class A amphipathic helix, which is a structural property that facilitates the ability to mimic the property of apoA-I to form small discrete discoidal particles via mixing with bilayer-forming lipids through cavitation, emulsification, or sonication. In an embodiment, wherein the apolipoprotein or the mimetic can be exchanged/displaced from PSLs by a second apolipoprotein or a second mimetic.

In an embodiment, the disclosure is directed to a method of delivering a therapeutically effective amount of a bioactive agent associated with PSL into a patient or subject is selected from the group generally applied to liposomes, consisting of aerosol administration, instillation administration, insufflation administration, intraperitoneal administration, intrathecal administration, intravenous administration, ocular administration, parenteral administration, sinus administration, transdermal administration, transmucosal administration, trans-serous administration, and a combination thereof. In an embodiment, the therapeutically effective bioactive agent, wherein the bioactive agent is formulated for controlled release.

In an embodiment, the disclosure is directed to a method of forming a metastable liposomal and lipid nanoparticle construct comprising flowing an aqueous phase in a channel wherein the aqueous phase comprises an apolipoprotein in an aqueous buffer solution; flowing a liquid phase in a second channel wherein the second fluid comprises a bioactive cargo that bears at least one hydrophobic region and a lipid component and a second lipid solvating fluid solution; mixing the aqueous phase and the lipid solvating phase utilizing microfluidic mixing; which results in a coalescing of the apolipoprotein with the cargo and the lipid component into a metastable liposomal and lipid nanoparticle construct, otherwise known as PSL. In an alternative embodiment, the bioactive cargo does not bear a hydrophobic domain, or it does not substantially bear a hydrophobic domain. In this alternative embodiment, the bioactive cargo may be combined in the aqueous or lipid solvating solutions. In this alternative embodiment, the bioactive cargo may have an affinity for the apolipoprotein, lipid, or additional elements in the preparation. In an embodiment, a preferred methodology employs a ratio of apolipoprotein to cargo/lipid of 1:150 w/w. PSLs become unstable when the ratio approaches about 1:200 w/w for apolipoprotein (or mimetic) to cargo/lipid. In an embodiment, the coalescing of apolipoprotein with the lipid/bioactive cargo is at a ratio between a range of about 1:100 to 1:200 w/w for apolipoprotein to lipid/bioactive cargo. In an embodiment, the ratio is at around 1:150 w/w for apolipoprotein to lipid/bioactive cargo. In an embodiment, the first channel and the second channel are in a microfluidic flow cell. In an embodiment, the aqueous phase comprises a saline or another aqueous buffer and wherein the liquid phase comprises an ethanol or another lipid-solvating solvent that is substantially miscible with the aqueous phase. In an embodiment, the lipid component is selected from the group consisting of a palmitoyl oleoyl phosphatidylcholine (POPC), a dimyristoylphosphatidylcholine (DMPC), a bilayer forming lipid, a non-bilayer forming lipids, a polyethylene glycol (PEG) conjugated lipid and a combination thereof. In an embodiment, the steps do not involve substantially shear force or cavitation.

In an embodiment, the apolipoprotein (i.e., the stabilizing protein) is present at relatively low quantity (e.g., 1:100 to 1:200 w/w for apolipoprotein to lipid/bioactive cargo) compared to other components found in extracts employed in PSL synthesis. The apolipoprotein and liposomal components are derived from extracts.

In a still further aspect, processes are provided for formulating bioactive agent into PSLs as described above. In one embodiment, the formulation process includes contacting a mixture that includes bilayer-forming lipids and a bioactive agent to form a lipid-bioactive agent mixture and contacting the lipid-bioactive agent mixture with a lipid binding polypeptide. In another embodiment, the formulation process includes formation of an emulsion of preformed bilayer-containing lipid vesicles to which a bioactive agent, dissolved in an appropriate solvent, is added. Appropriate solvents for solubilizing a bioactive agent for this procedure include solvents with polar or hydrophilic character that are capable of solubilizing a bioactive agent to be incorporated into a PSL of the invention and is miscible in an aqueous solution. Examples of suitable solvents include, but are not limited to, acetone, acetonitrile, dimethylsulfoxide (DMSO), dimethylformamide (DMF), ethanol, glycerol, isopropanol, methanol, propanol, pyridine, and tetrahydrofuran. To the lipid/bioactive agent mixture, apolipoproteins are added using microfluidic mixing processes.

Chimeric lipoproteins are often created by chemically conjugating lipoprotein to modifying moiety. Apolipoproteins typically contain a variety of functional groups, e.g., carboxylic acid (—COOH), free amino (NH), or sulfhydryl (SH) groups, that are available for reaction with a suitable functional group on the modifying moiety or on a linker to bind the modifying moiety thereto. A modifying moiety may be attached at the N-terminus, the C-terminus, or to a functional group on an interior residue (i.e., a residue at a position intermediate between the N- and C-termini) of an apolipoprotein molecule. Alternatively, the apolipoprotein and/or the moiety to be tagged can be derivatized to expose or attach additional reactive functional groups.

In some embodiments, apolipoprotein fusion proteins that include a polypeptide modifying moiety are synthesized using recombinant protein expression systems. Typically, this involves creating a nucleic acid (e.g., DNA) sequence that encodes the apolipoprotein and the modifying moiety such that the two polypeptides will be in frame when expressed, placing the DNA under the control of a promoter, expressing the protein in a host cell, and isolating the expressed protein. Chimeric apolipoprotein DNA sequences encoding apolipoprotein and modifying moieties, as described herein, may be cloned or amplified by in vitro methods, such as, for example, the polymerase chain reaction (PCR), the ligase chain reaction (LCR), the transcription-based amplification system (TAS), or the self-sustained sequence replication system (SSR). A wide variety of cloning and in vitro amplification methodologies are well known to persons of skill. Examples of techniques sufficient to direct persons of skill through in vitro amplification methods are found for example, in Mullis et al., (1987) U.S. Pat. No. 4,683,202, PCR Protocols A Guide to Methods and Applications (Innis et al. eds) Academic Press Inc. San Diego, Calif. (1990) (Innis); Arnheim & Levinson (Oct. 1, 1990) C&EN 36-47. The Journal of NIH Research (1991) 3: 81-94: (Kwoh et al. (1989) Proc. Natl. Acad. Sci. USA 86: 1173; Guatelli et al. (1990) Proc. Natl. Acad. Sci. USA 87, 1874; Lomeli et al. (1989). J. Clin. Chem., 35: 1826; Landegren et al., (1988) Science, 241: 1077-1080; Van Brunt (1990) Biotechnology, 8: 291-294: Wu and Wallace, (1989) Gene, 4:560; and Barringer et. al.. (1990) Gene, 89: 117. In addition, DNA encoding desired fusion protein sequences may be prepared synthetically using methods that are well known to those of skill in the art, including, for example, direct chemical synthesis by methods such as the phosphotriester method of Narang et al. (1979) Meth. Enzymol. 68:90-99, the phosphodiester method of Brown et al., (1979) Meth. Enzymol. 68: 109-151, the diethylphosphoramidite method of Beaucage et al. (1981) Tetra. Lett., 22: 1859-1862, or the solid support method of U.S. Pat. No. 4,458,066.

In some embodiments of the invention, an apolipoprotein is provided that has been modified such that when the polypeptide is incorporated into a bioactive agent PSL as described above, the modification will increase particle stability, confer targeting ability, or enhance the bioactivity of the cargo. In some embodiments, the modification permits the apolipoproteins of a PSL to stabilize the PSL's structure or conformation. In one embodiment, the modification includes introduction of cysteine residues into apolipoprotein molecules to permit formation of intramolecular or intermolecular disulfide bonds, e.g., by site-directed mutagenesis. In another embodiment, a chemical crosslinking agent is used to form intermolecular links between apolipoprotein molecules to modify the structural flexibility of the apolipoprotein and thereby modulate the stability of the PSLs. Intermolecular crosslinking prevents or reduces dissociation of apolipoprotein molecules from particles and/or prevents displacement by apolipoprotein molecules within an individual to whom the PSLs are administered. In other embodiments, an apolipoprotein is modified either by chemical derivatization of one or more amino acid residues or by site directed mutagenesis, to confer targeting ability to or recognition by a cell surface receptor.

The invention provides a delivery system for delivery of a bioactive agent to an individual, comprising bioactive agent PSLs as described above and a carrier, optionally a pharmaceutically acceptable carrier. In some embodiments, the delivery system comprises an effective amount of the bioactive agent.

The invention provides methods for administering a bioactive agent to an individual. The methods of the invention include administering a PSL, as described above that includes an apolipoprotein, at least one lipid bilayer, and a bioactive agent, wherein the interior of the PSL may encapsulate an aqueous internal compartment. Optionally, a therapeutically effective amount of the PSLs is administered, optionally in a pharmaceutically acceptable carrier. Typically, the bioactive agent includes at least one hydrophobic region, which may be integrated into a hydrophobic region of the lipid bilayer. The route of administration may vary according to the nature of the bioactive agent to be administered, the individual, or the condition to be treated. Where the individual is a mammal, generally administration is parenteral. Routes of administration include those commonly applied to liposomes, but are not limited to intramuscular, intraocular, intraperitoneal, intrathecal, intravenous, parenteral, subcutaneous, topical, transdermal, transmucosal, transnasal, and transpulmonary. In one embodiment, the PSLs are administered as an aerosol. PSLs may be formulated in a pharmaceutically acceptable form for administration to an individual, optionally in a pharmaceutically acceptable carrier or excipient. The invention provides pharmaceutical compositions in the form of PSLs in a solution for parenteral administration. For preparing such compositions, methods well known in the art may be used, and any pharmaceutically acceptable carriers, diluents, excipients, or other additives normally used in the art may be used. The PSLs of the present invention can be made into pharmaceutical compositions by combination with appropriate medical carriers or diluents. For example, PSLs can be combined with solvents commonly used in the preparation of injectable solutions, such as for example, physiological saline, water, or aqueous dextrose. Other suitable pharmaceutical carriers and their formulations are described in Remington's Pharmaceutical Sciences, 1965. Such formulations may be made up in sterile vials containing PSLs and optionally an excipient in a dry powder or lyophilized powder form. Prior to use, the physiologically acceptable diluent is added, and the solution withdrawn via syringe for administration to an individual.

PSLs may be administered according to the methods described herein to treat several conditions including, but not limited to, bacterial infections, cancer, cardiovascular disease, fungal infections, genetic disease, hormonal disorder, immune disorder, inflammatory disorder, metabolic disorders, neurological disorder, nutritional disorder, toxicity/poisoning, and wound healing. PSLs may be used, for example in the delivery of medication prophylactically, for example to prevent a bacterial or fungal infection (e.g., pre- or post-surgically) or as a carrier of a vaccine. PSLs may be used, for example, to deliver an anti-tumor agent (e.g., chemotherapeutic agent, radionuclide) to a tumor. The method includes administering a therapeutically effective amount of a chemotherapeutic agent in bioactive agent-containing PSLs, as described above, in a pharmaceutically acceptable carrier. In one embodiment, the chemotherapeutic agent is camptothecin. In one embodiment, the apolipoprotein includes a moiety that targets the particle to a particular tumor. PSLs may also be used for administration of nutraceutical substances, i.e., a food or dietary supplement that provides health benefits. In some embodiments, PSLs are co-administered with other conventional therapies, for example, as part of a multiple drug “cocktail” or in combination with one or more orally administered agents, for example, for treatment of a fungal infection. PSLs may also be administered as carriers of insecticides or herbicides.

A PSL of the invention may include a targeting functionality, for example to target the PSLs to a particular cell or tissue type, or to the infectious agent itself. In some embodiments, the PSL includes a targeting moiety attached to apolipoprotein, lipid component, or bioactive agent cargo. In some embodiments, the bioactive agent cargo has a targeting capability. In some embodiments, the lipid component of PSLs may be chemically modified have a targeting capability. In some embodiments, by engineering receptor recognition properties into the apolipoprotein, PSLs can be targeted to a specific cell surface receptor. For example, PSLs may be targeted to a particular cell type, for example by modifying the apolipoprotein component of the PSL to render it capable of selectively binding to a cell type-specific receptor on the surface of the cell type being targeted. In some embodiments, the PSL may target cells that are the locus of infection or diseased. In one example, a receptor-mediated targeting strategy may be used to deliver antileishmanial agents to macrophages, which are the primary site of infection for protozoal parasites from the genus Leishmania. Example species include Leishmania major, Leishmania donovani, and Leishmania braziliensis. PSLs containing an antileishmanial agent may be targeted to macrophages by altering the apolipoprotein component of the PSLs to confer recognition by the macrophage endocytic Class A Scavenger Receptor (SR-A). For example, an apolipoprotein which has been chemically or genetically modified to interact with SR-A may be incorporated into PSLs that contain one or more bioactive agents that are effective against Leishmania species, such as, for example, amphotericinB (AmB), a pentavalent antimonial, and/or hexadecylphosphocholine. Targeting of PSLs that contain an antileishmanial agent specifically to macrophages may be used as a means of inhibiting the growth and proliferation of Leishmania spp. In one embodiment an SR-A targeted PSL containing AmB is administered to an individual in need of treatment for a leishmanial infection. In another embodiment, another antileishmanial agent, such as hexadecylphosphocholine is administered prior, concurrently, or subsequent to treatment with the AmB containing-PSLs. In some embodiments, targeting is achieved by modifying the apolipoprotein incorporated into the PSL, thereby conferring SR-A binding ability to the PSL. In some embodiments, targeting is achieved by altering the charge density of the apolipoprotein by chemically modifying one or more lysine residues, for example with malondialdehyde, maleic anhydride, or acetic anhydride at alkaline pH (see, e.g., Goldstein et al. (1979) Proc. Natl. Acad. Sci. 98:241-260). In other embodiments, an apolipoprotein molecule, such as any of the apolipoproteins described herein may also be chemically modified by, for example acetylation or maleylation, and incorporated into a PSL containing an antileishmanial agent. In other embodiments, SR-A binding ability is conferred to a PSL by modifying the apolipoprotein component by site directed mutagenesis to replace one or more positively charged amino acids with a neutral or negatively charged amino acid.

In the preferred embodiment, PSLs are prepared using microfluidic processes. In an embodiment, the first channel and the second channel are in a microfluidic flow cell, wherein one channel is aqueous and the other channel is lipid solvating. In the preferred embodiment, the apolipoprotein component is mixed into an aqueous solution and the lipid component and bioactive cargo are mixed into a lipid solvating solution. In an embodiment, the aqueous channel comprises a saline or another aqueous buffer and the lipid solvating channel phase comprises an ethanol or another lipid-solvating solvent that is substantially miscible with the aqueous phase. In an embodiment, the lipid component is selected from the group consisting of, but are not limited to, dipalmitoyl phosphatidylcholine (DMPC), dimyristoyl phosphoglycerol (DMPG), palmitoyl oleoyl phosphatidylcholine (POPC), dipalmitoyl phosphatidylcholine (DPPC), dipalmitoyl phosphatidylserine (DPPS), dipalmitoyl phosphatidylglycerol (DPPG), distearoyl phosphatidylglycerol (DSPG), egg yolk phosphatidylcholine (egg PC), soy bean phosphatidyl choline, phosphatidylinositol, phosphatidic acid, sphingomyelin, and cationic phospholipids. Examples of other suitable bilayer-forming lipids include cationic lipids, glycolipids, polyethylene glycol (PEG) conjugated lipid and a combination thereof. Examples of suitable lipid solvents include, but are not limited to, acetone, acetonitrile, dimethylsulfoxide (DMSO), dimethylformamide (DMF), ethanol, glycerol, isopropanol, methanol, propanol, pyridine, and tetrahydrofuran. To the lipid-bioactive agent mixture, lipoproteins are added using lamellar flow microfluidic processes. In some embodiments the bioactive cargo is chemically bound to the apolipoprotein component of PSLs and mixed into the aqueous solution. In the preferred embodiment, the two channels are mixed by microfluidic lamellar flow mixing, which is a non-turbulent or low turbulence mixing process and facilitates the production of large lipid particles. During microfluidic mixing, the resultant combined solution consisting of apolipoprotein, lipid component, and bioactive cargo coalesce into PSL particles. In an embodiment, the flow path of the microfluidic device contains turns, deviations, and crenellations that yield a non-linear flow path. These elements facilitate mixing by introducing a flow velocity gradient across a cross section of the flow stream. This introduces microturbulence by causing changes in the velocity of one lamellar flow channel relative to another. In an embodiment, a preferred methodology employs a ratio of apolipoprotein to lipid/bioactive cargo of 1:150 w/w. PSLs become unstable when the ratio approaches about 1:200 w/w for apolipoprotein to lipid/bioactive cargo. In an embodiment, the ratio of apolipoprotein to lipid/bioactive cargo is between about 1:100 to 1:200 w/w. In an embodiment, the ratio is at approximately around 1:150 w/w for apolipoprotein to lipid/bioactive cargo. In an embodiment, the steps do not involve substantially shear force or cavitation. In an embodiment, the lipid solvating solution containing bioactive cargo is the product of lipid emulsion prepared by sonication methods known in the art of lipid emulsion preparation.

In an embodiment, PSLs are prepared by incubation of solvated lipids/bioactive cargo with apolipoprotein. In an embodiment, the lipid component, as listed above, are dispersed in miscible solvent, as listed above, by agitation or sonication. To the solubilized lipid component, bioactive agent is added to form a lipid/bioactive agent mixture. In some embodiments, the solvent is volatile or dialyzable for convenient removal after PSL formation. Apolipoprotein in aqueous solvent is added to the solvated lipid/bioactive agent and mixed by agitation and/or sonication. Typically, the lipid/bioactive agent mixture and apolipoprotein are combined and incubated at or near the gel to liquid crystalline phase transition temperature of the particular bilayer forming lipid or lipid/bioactive agent mixture being used. The phase transition temperature may be determined by calorimetry. Preferably, a suitable lipid/bioactive agent mixture composition is used such that, upon dispersion in aqueous media, the lipid vesicles that are present or are formed provide a suitable environment to transition a bioactive agent from a carrier solvent into an aqueous milieu, without precipitation or phase separation of the bioactive agent. The pre-formed lipid bilayer vesicles are also preferably capable of undergoing apolipoprotein transformation to form the PSLs of the invention. Further, the lipid/bioactive agent complex preferably retains properties of the lipid vesicles that permit transformation into PSLs upon incubation with an apolipoprotein under appropriate conditions. The unique combination of lipid/bioactive agent mixture and apolipoprotein properties combine to create a system whereby, under appropriate conditions of pH, ionic strength, temperature, and lipid component, bioactive agent and apolipoprotein concentration, a ternary structural reorganization of these materials occurs resulting in the formation of a metastable PSL particle assembly with a bioactive agent incorporated into the lipid milieu of the bilayer. The PSLs prepared by any of the above processes may be further purified, for example by dialysis, density gradient centrifugation and/or gel permeation chromatography.

In a preparation method for formation of bioactive agent-containing PSLs, the percent of the bioactive agent used in the procedure that is incorporated into PSLs is preferably at least about 70, more preferably at least about 80, even more preferably at least about 90, even more preferably at least about 95. The invention provides bioactive agent-containing PSLs prepared by any of the above methods. In one embodiment, the invention provides a pharmaceutical composition comprising a PSL prepared by any of the above methods and a pharmaceutically acceptable carrier.

Several methods have been utilized to produce liposomes. Because vesiculation of natural phospholipid bilayers is not a spontaneous process, physical and chemical methods are used to produce well-defined liposomes from hydrated lipids. Usually, these methods require the input of high energy (e.g., ultrasonic treatment, high pressure, and/or elevated temperatures) to disperse low critical micelle concentration phospholipids as a metastable liposome phase.

One method for the preparation of liposomes involves the solvent evaporation of an oil-in-water emulsion. The oil phase contains one or more pharmaceutical agents or bioactive agents, cholesterol and lipids and the aqueous phase contains an emulsifier. An emulsion consists of two immiscible liquids (usually oil and water), with one of the liquids dispersed as small spherical droplets in the other. A system that consists of water droplets dispersed in an oil phase is called a water-in-oil or W/O emulsion (e.g., margarine, butter, and spreads). The process of converting two separate immiscible liquids into an emulsion, or of reducing the size of the droplets in a preexisting emulsion, is known as homogenization.

In liposomes used for drug delivery, the breakdown of the vesicle structure of the compositions has been observed. The term “emulsion stability” is broadly used to describe the ability of an emulsion to resist changes in its properties with time. Emulsions may become unstable through a variety of physical processes including creaming, sedimentation, flocculation, coalescence, and phase inversion. Creaming and sedimentation are both forms of gravitational separation. Creaming describes the upward movement of droplets due to the fact that they have a lower density than the surrounding liquid, whereas sedimentation describes the downward movement of droplets due to the fact that they have a higher density than the surrounding liquid. Flocculation and coalescence are both types of droplet aggregation. Flocculation occurs when two or more droplets come together to form an aggregate in which the droplets retain their individual integrity, whereas coalescence is the process where two or more droplets merge together to form a single larger droplet. Extensive droplet coalescence can eventually lead to the formation of a separate layer of oil on top of a sample, which is known as “oiling off.”

Thermodynamics are largely responsible for the separation of phases. If an emulsion is generated by homogenizing pure oil and pure water together, the two phases will rapidly separate into a system that consists of a layer of oil (lower density) on top of a layer of water (higher density). This is because droplets tend to merge with their neighbors, which eventually leads to complete phase separation. The disruption of liposome structure over time and premature drug leakage present significant, and potentially very hazardous, problems for using liposomes as vehicles for drug delivery. Drug leakage from liposomes during long term storage, lyophilization and reconstitution can decrease the predictability and increase the toxicity of drug delivery using liposomes. Specifically, the premature release and leakage of the drug from the liposome results in a faster distribution of the drug in the plasma component, increased toxicity, and decreased concentrations of the drug released at the tumor site. Thus, a need exists to improve liposome design to increase liposome stability and eliminate premature drug leakage.

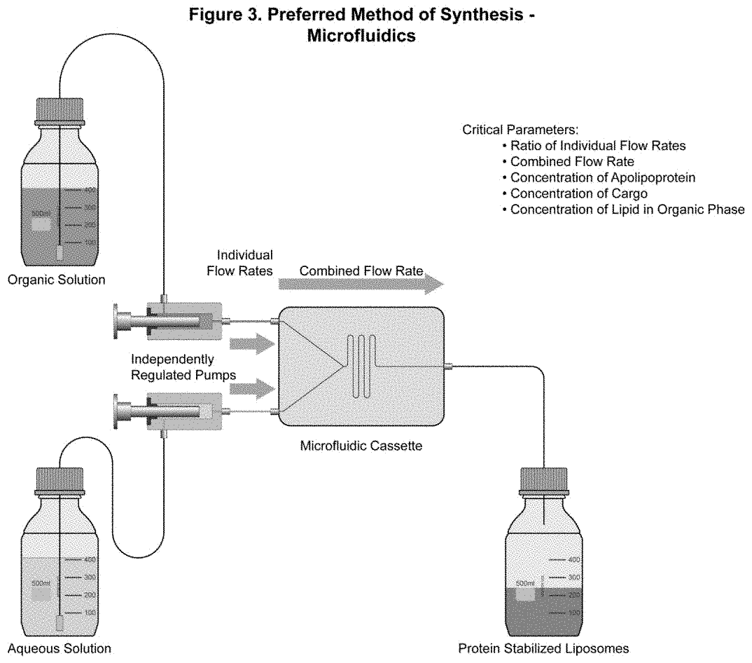

The present invention provides microfluidic systems for producing PSLs by nanoprecipitation using controlled mixing of polymeric solutions in a fluid that is not a solvent for the polymer (i.e., a non-solvent such as water) (FIG. 3). The mixing can be achieved by any techniques or mixing apparatus known in the art of microfluidics, including, but not limited to, hydrodynamic flow focusing. The present invention provides microfluidic devices for producing polymeric drug PSLs. In general, a microfluidic flowcell device comprises at least two channels that converge into a mixing apparatus. In some embodiments, the channels join at an angle ranging between zero degrees and 180 degrees. A stream of fluid can flow through each channel, and the streams join and flow into the mixing apparatus. In general, at least one stream comprises a lipid solubilizing solution, and at least one stream comprises an aqueous solvent. In some embodiments, the flow of the streams is laminar or substantially non-turbulent in other embodiments, the flow streams are combined via micro turbulent flow mixing or Dean vortex mixing.

In some embodiments, the channels have a circular cross-section. In some embodiments, the channels that converge into the mixing apparatus are of uniform shape. In some embodiments, the width or height of each channel ranges from approximately 1 μm to approximately 1000 μm. In some embodiments, the length of each channel ranges from approximately 100 μm to approximately 10 cm. Channels may be composed of any material suitable for the flow of fluid through the channels. Typically, the material is one that is resistant to solvents and non-solvents that are used in the preparation of particles. In general, the material is not one that will dissolve or react with the solvent or non-solvent. In some embodiments, channels are composed of glass, silicon, metal, metal alloys, polymers, plastics, photocurable epoxy, ceramics, or combinations thereof. In some embodiments, channels are formed by lithography, etching, embossing, or molding of a polymeric surface. In general, the fabrication process may involve one or more of any of the processes described herein, and different parts of a device may be fabricated using different methods and assembled or bonded together.

Typically, a source of fluid is attached to each channel, and the application of pressure to the source causes the flow of the fluid in the channel. The pressure may be applied by a syringe, a pump, and/or gravity. In some embodiments, the applied pressure is regulatable (i.e., the applied pressure may be increased, decreased, or held constant). In some embodiments, the flow rate is regulatable by adjusting the applied pressure. In some embodiments, the flow rate is regulatable by adjusting the size (e.g., length, width, and/or height) of the channel. In some embodiments, the flow rate may range from 0.1 ml/min to 10.0 ml/min. In specific embodiments, the flow rate is approximately 10 ml/min for non-solvent solutions (e.g., an aqueous solution such as water). In specific embodiments, the flow rate is approximately 2.0 ml/min for solvent solutions (e.g., polar solvent solutions like ethanol, isopropanol, and methanol).

The present invention provides microfluidic systems in which a plurality of inlet streams of solutions with polymer, targeting moieties, lipids, drug, payload, etc. converge and mix, and the resulting mixture yields a stream which joins a stream of non-solvent and flows into the mixing apparatus. In some embodiments, a microfluidic system may comprise at least 2, at least 3, at least 4, at least 5, at least 6, at least 7, at least 8, at least 9, at least 10, or more inlet streams. The flow of each inlet stream is regulated by a source of fluid, wherein the application of pressure to the source causes the flow of fluid in the inlet stream. In some embodiments, the same amount of pressure is applied to all of the channels and/or inlet streams. In some embodiments, different amounts of pressure are applied to different channels and/or inlet streams. Thus, in some embodiments, the flow rate may be the same through all channels and/or inlet streams, or the flow rate may be different in different channels and/or inlet streams. In some embodiments, the flow path of the microfluidic device contains turns, deviations, and crenellations that yield a non-linear flow path. In some embodiments the flow path is a series of switchbacks or right angle turns or overlapping crisscross pathways. Other pathways include Tesla valves or similar structures or other flow geometries that yield micro turbulent flow mixing such as Staggered Herringbone Mixers or Dean Vortexing Bifurcating Mixers. These and other microfluidic mixing processes yield gentle mixing that supports the formation of large liposomal constructs that would normally be disassembled by the shear forces imparted by the traditional liposome synthesis methodologies involving cavitation and extrusion.

PSLs of the invention are stable for long periods of time under a variety of conditions. Particles, or compositions comprising particles of the invention, may be stored at room temperature, refrigerated (e.g., about 4° C.), or frozen (e.g., about −20° C. to about −80° C.). They may be stored in solution or dried (e.g., lyophilized). PSLs may be stored in a lyophilized state under inert atmosphere, frozen, or in solution at 4° C. Particles may be stored in an aqueous liquid medium, such as a buffer (e.g., phosphate or other suitable buffer), or in a carrier, such as for example a pharmaceutically acceptable carrier, for use in methods of administration of a bioactive agent to an individual. Alternatively, PSLs may be stored in a dried, lyophilized form and then reconstituted in liquid medium prior to use.

The reagents and PSLs described herein can be packaged in kit form. In one aspect, the invention provides a kit that includes PSLs formulated with bioactive cargo in concentrated or market-ready state, in suitable pharmaceutically acceptable carrier(s), packaging, and instructions for manufacture or use. Kits of the invention include any of the following, PSLs with or without incorporated bioactive cargo, unincorporated bioactive agents, pharmaceutically acceptable carrier solutions/formulations, and packaging for administration to an individual. Each reagent or formulation is supplied in suitable packaging in liquid buffer as a concentrate or a state ready for administration to an individual or suitable for inventory storage or addition into a reaction or secondary manufacturing process. Additionally, kits optionally include labeling and/or instructional or interpretive materials providing directions (i.e., protocols) for the use of PSLs. While the instructional materials typically comprise written or printed materials they are not limited to these formats. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to Internet sites that provide such instructional materials. In addition, the kit could be part of a larger kit or manufacturing process of a pharmaceutical or biological agent.

The following example of making PSLs is intended to illustrate but not limit the invention.

### Example 1: Cannabinoid-PSL Manufacture

### Scope and Application

In the following example, a method is provided that yields PSLs that contain cannabis/hemp extract (other bioactive agents can be substituted). The product is in concentrated form, which is suitable for subsequent dilution for liquid formulation, gel formulation, nasal spray, tincture, and sports gel formulation. The consistency of this method is greater than 80%, as determined by dynamic light scatter particle size analysis.

### Procedure

The initial step is to prepare the microfluidic device (flow cell) for PSL production. The microfluidic device is as described above and consists of two inlet channels, one outlet channel and a microfluidic flow path of 0.3 mm width, a length of approximately 3.0 cm, wherein the two inlet channels intersect and direct the combined fluid through a pathway that contains at least 5 switchbacks or 90° turns, and staggered herringbone crenelations on at least one flow surface. The procedure involves two single channel syringe pumps or one dual channel syringe pump. At scale, the syringe pump(s) can be substituted for FPLC or HPLC pumps capable of differentially/independently pumping at least two liquids simultaneously. Speed/flow rates and the ratio of the volumes discussed here can be directly translated to a FPLC or HPLC mediated procedure. First, enter the dimensions or preset settings for the syringes to be used (e.g., BD Plastic 5 mL syringe) into the syringe pump(s). Fill the channel A syringe with 3 mL sterile deionized water (H₂O) and fill the channel B syringe with 3 ml>95% ethanol (EtOH). Connect the syringes to PEEK tubing (or other medical grade medium pressure compatible tubing, typically used in HPLC processes) that are attached to appropriate channels on the microfluidic device and attach a similar PEEK tubing to the microfluidic device outlet and position the other end of this PEEK tubing into an appropriate container that will receive waste product. Set the syringe pump(s) to 2.5 mL and flow rate to 5.5 mL/min for both channels. The combined flow rate for both channels should not be greater than 12 mL/min or it could damage the microfluidic device, compromising yield and consistency of PSL particle size. Activate the syringe pump(s) simultaneously. When the pumps have completed the passage process, move on to the next step.

Fill the channel A syringe with 3 mL sterile 1×PBS and fill the channel B syringe with 3 mL>95% ethanol. Using the same volume and flow rate settings as the prior step, activate the syringe pump(s) simultaneously. When the pumps have completed the passage process, move on to the next step.

The next step is the synthesis of PSL concentrate. The following procedure assumes an apolipoprotein concentration of 0.2 mg/m L. Refer to Table 1 if other concentrations of apolipoprotein are used. The method has been validated for apolipoprotein concentrations as low as 0.1 mg/mL and as high as 2.0 mg/mL, but optional results are obtained with apolipoprotein at concentrations between 0.2 to 0.5 mg/mL. Program the syringe pump(s), to the syringe brand used and for channel A a volume of 20 mLs and a flow rate of 10 mL/min and channel B to a volume of 5 mLs and flow rate to 1 mL/min. If a concentration of apolipoprotein other than 0.2 mg/mL is used, adjust the volumes and flow rates as per Table 1. Fill the channel A 20 mL syringe with apolipoprotein (at 0.2 mg/mL). Fill the channel B 5 mL syringe with 30% Cannabinoid distillate (in EtOH; in this method cannabinoid could be replaced with any ethanol soluble bioactive agent). Position the outlet end of the PEEK tubing that had been in the waste receiving container into an appropriate container that will receive the PSL product. Activate the syringe pump(s) simultaneously. When the pumps have completed the passage process, move on to the next step.

TABLE 1
Ratios of Apolipoprotein to Cannabinoid Distillate

| | | | Cannabinoid Distillate (30%; other | |
|---|---|---|---|---|
| Apolipoprotein | Apolipoprotein | | bioactive agent could be used) | |
| Concentration | Volume | Flow Rate | Volume | Flow Rate |
| (mg/mL) | (mL) | (mL/min) | (mL) | (mL/min) |
| 0.50 | 20 | 8.80 | 5.00 | 2.20 |
| 0.45 | 20 | 8.98 | 4.50 | 2.02 |
| 0.40 | 20 | 9.17 | 4.00 | 1.83 |
| 0.35 | 20 | 9.36 | 3.50 | 1.64 |
| 0.30 | 20 | 9.57 | 3.00 | 1.43 |
| 0.25 | 20 | 9.78 | 2.50 | 1.22 |
| 0.20 | 20 | 10.00 | 2.00 | 1.00 |

NOTE:
The ratios in Table 1 will result in different concentrations of distillate. This differential will be adjusted for in the conversion of concentrate to final product, defined in Table 1.

The PSL concentrate can be used in the formulation of an array of products. These are summarized in Table 2. To create a liquid cannabinoid PSL, the PSL concentrate is combined with saline at a ratio of 1:20 for a starting concentration of apolipoprotein at 0.2 mg/mL. The liquid formulation may be subjected to filter sterilization techniques, which would render it compatible with oral, parenteral, and intravenous modes of administration. For the Gel formulations, PSL concentrate is combined with a commercially available aloe vera and gelling agent like carbomer, methyl cellulose, xanthan gum, or other gelling agent commonly used in cosmetic or pharmaceutical formulations.

TABLE 2
Dilution Ratio of Product from Cannabinoid
Distillate - PSL Concentrate

| An Initial | Distillate | | | |
|---|---|---|---|---|
| Concentration | Concentration | | | |
| (mg/mL) | (%) | Liquid | Gel | Sports Gel |
| 0.50 | 6.00 | 43.96 | 54.95 | 137.36 |
| 0.45 | 5.51 | 40.37 | 50.46 | 126.14 |
| 0.40 | 5.00 | 36.63 | 45.79 | 114.47 |
| 0.35 | 4.47 | 32.75 | 40.93 | 102.34 |
| 0.30 | 3.91 | 28.64 | 35.81 | 89.51 |
| 0.25 | 3.33 | 24.40 | 30.49 | 76.24 |
| 0.20 | 2.73 | 20.00 | 25.00 | 62.50 |

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes and to the same extent as if each individual publication, patent, or patent application were specifically and individually indicated to be so incorporated by reference.

### REFERENCES

Arnheim & Levinson (Oct. 1, 1990) C&EN 36-47.
Barringer et al. (1990) Gene, 89: 117.
Beaucage et al. (1981) Tetra. Lett., 22: 1859-1862.
Brown et al., (1979) Meth. Enzymol. 68: 109-151.
Goldstein et al. (1979) Proc. Natl. Acad. Sci. 98:241-260.
Guatelli et al. (1990) Proc. Natl. Acad. Sci. USA 87, 1874.
Jackson, et al. (1970). “Cytotoxic Threshold of Vincristine in a Murine and a Human Leukemia Cell Line In Vitro”, Cancer Research 39: 4346-4349.
The Journal of NIH Research (1991) 3: 81-94.
Kwoh et al. (1989) Proc. Natl. Acad. Sci. USA 86: 1173.
Fullerton S M, Buchanan A V, Sonpar V A, Taylor S L, Smith J D, Carlson C S, et at (June 2004). “The effects of scale: variation in the APOA1/C3/A4/A5 gene duster” Human Genetics, 115 (1): 36-56.
Landegren et al., (1988) Science, 241: 1077-1080.
Lomeli et al. (1989). J. Clin. Chem., 35: 1826.
Mullis et al. (1987). “Specific synthesis of DNA in vitro via a polymerase-catalyzed chain reaction” Methods Enzymol, 155: 335-50.
Narang et al. (1979) Meth. Enzymol. 68:90-99.
PCR Protocols A Guide to Methods and Applications (Innis et al. eds) Academic Press Inc. San Diego, Calif. (1990) (Innis).
Remington's Pharmaceutical Sciences (1965).
U.S. Pat. No. 4,458,066.
U.S. Pat. No. 4,683,202.
Van Brunt (1990) Biotechnology, 8: 291-294.
Wu and Wallace, (1989) Gene, 4:560.

What is claimed:

1. A synthetic protein stabilized liposome (PSL) comprises:
an apolipoprotein, wherein the apolipoprotein comprises a natural apolipoprotein, an apolipoprotein mimetic, an apolipoprotein variant, an apolipoprotein chimera, or a combination thereof;
a bioactive agent;
a liposome forming lipid;
wherein the synthetic PSL is configured to have a diameter of or greater than about 60 nm; and
wherein the synthetic PSL is configured to transport the bioactive agent across a cellular barrier selected from the group consisting an endothelium, an epithelium, a mucous membrane, a serous membrane, and a combination thereof.

2. The synthetic PSL of claim 1, wherein a final concentration of the apolipoprotein relative to the volume of the PSL in a solution is between about 0.1 μg/mL to about 11 mg/mL by weight.

3. The synthetic PSL of claim 2, wherein the final concentration of the apolipoprotein relative to the volume of the PSL in the solution is between about 0.5 μg/mL to about 200 μg/mL.

4. The synthetic PSL of claim 1, wherein the PSL is configured to have a diameter from about 60 nm to about 3.5 μm.

5. The synthetic PSL of claim 1, wherein the PSL is configured to have a diameter over about 2.0 μm.

6. The synthetic PSL of claim 1, wherein the PSL is configured to be substantially stabilized when stored at about 4° C. for at least about one year or when stored at room temperature for about three months and wherein substantial stability means at least about 80% of the PSLs are not substantially disassembled for about one year at about 4° C. or for about three months at room temperature.

7. The synthetic PSL of claim 1, wherein the PSL is configured to be substantially stabilized when stored at about 4° C. for at least about two years or when stored at room temperature for about six months and wherein substantial stability means at least about 80% of the PSLs are not substantially disassembled for about two years at about 4° C. or for about six months at room temperature.

8. The synthetic PSL of claim 1, wherein the PSL substantially disassembles at about 65° C.

9. The synthetic PSL of claim 1, wherein the apolipoprotein mimetic is configured to form substantially a class A amphipathic helix or substantially mimics an ability of apoA-I to form discoidal synthetic nascent High Density Lipoproteins.

10. The synthetic PSL of claim 1, wherein the apolipoprotein is exchanged with a second apolipoprotein.

11. The synthetic PSL of claim 1 wherein the apolipoprotein comprises an exchangeable apolipoprotein configured to displace an initial apolipoprotein from a preformed PSL.

12. The synthetic PSL of claim 11, wherein the exchangeable apolipoprotein comprises a motif of approximately at least about 22 amino acids configured to form an amphipathic alpha helix.

13. The synthetic PSL of claim 12, wherein the exchangeable apolipoprotein comprises a plurality of the motif of at least about 22 amino acids configured to form the amphipathic alpha helix.

14. (canceled)

15. The synthetic PSL of claim 12, wherein the exchangeable apolipoprotein comprises a peptide configured to mimic an amphipathic helical domain of the apolipoprotein, wherein a plurality of positively charged residues are configured at the polar-nonpolar face interface and a plurality of negatively charged residues configured at the center of the polar face.

16. The synthetic PSL of claim 12, wherein the exchangeable apolipoprotein is selected from the group consisting of (1) apoA-II, C-I, C-II, and C-III with an amphipathic helical domain; (2) apoA-I and apo-A-E with an amphipathic helical domain; and (3) apoA-IV with an amphipathic helical domain.

17. The synthetic PSL of claim 1, wherein the synthetic PSL is among a plurality of PSLs and wherein the plurality of PSLs is substantially stable with at least about 80% of the PSLs retaining functionality or secondary structure composition.

18. The synthetic PSL of claim 17, wherein the substantially stable PSLs are determined by a stability analysis comprises showing whether at least about 80% of the synthesized PSLs bear identity with a predefined particle distribution profile comprising a substantially consistent distribution of the bioactive agent in the PSLs or a substantially consistent distribution of particle size for the PSLs.

19. The synthetic PSL of claim 17, wherein the substantially stable PSLs comprises maintaining the integrity of at least about 80% of PSLs at room temperature for at least two days.

20. The synthetic PSL of claim 19, wherein the substantially stable PSLs comprises at least about 80% of the PSLs substantially lacking a separation of the bioactive agent cargo from the PSL.

21. The synthetic PSL of claim 19, wherein the substantially stable PSLs comprises at least about 80% of the PSLs comprises lacking substantially an appearance of a two-phase solution for the PSLs.

22. (canceled)

23. The synthetic PSL of claim 1, wherein the apolipoprotein is selected from the group consisting of an apolipoprotein A-I, apolipoprotein A-II, apolipoprotein A-IV, apolipoprotein A-V, apolipoprotein C-I, apolipoprotein C-II, apolipoprotein C-III, apolipoprotein D, apolipoprotein E, apolipoprotein H, apolipoprotein J, apolipoprotein M, or fragments, natural variations, an isoform, an amino acid substitution variant, an analog, a chimeric form, a modified form thereof, and a combination thereof.

24. (canceled)

25. (canceled)

26. (canceled)

27. (canceled)

28. (canceled)

29. The synthetic PSL of claim 1, wherein the bioactive agent is selected from the group consisting of a protein, a nucleic acid, a chemical, a small molecule, a bioactive lipid, and a combination thereof.

30. (canceled)

31. (canceled)

32. (canceled)

33. A method of forming a metastable liposomal and lipid nanoparticle construct comprises:
a. flowing an aqueous phase in a first channel wherein the aqueous phase comprises an apolipoprotein in a first fluid solution;
b. flowing a liquid phase in a second channel wherein the second fluid comprises a bioactive agent and a lipid component in a second fluid solution;
c. mixing the aqueous phase and the liquid phase by utilizing a microfluidic lamellar flow between the two phases; and
d. coalescing the apolipoprotein with the bioactive agent and the lipid component into the metastable liposomal and lipid nanoparticle construct otherwise referred to as PSL.

34. The method of forming the metastable liposomal and lipid nanoparticle construct of claim 33; wherein the coalescing the apolipoprotein with the cargo and the lipid component is at a ratio between a range of about 1:50 to about 1:300 w/w for apolipoprotein to bioactive agent and lipid component.

35. The method of forming the metastable liposomal and lipid nanoparticle construct of claim 33; wherein the ratio is at around 1:150 w/w for apolipoprotein to bioactive agent and lipid component.

36. The method of forming the metastable liposomal and lipid nanoparticle of claim 33, wherein the apolipoprotein in a solution comprises a final concentration of between about 0.1 μg/mL to about 11 mg/mL by weight.

37. The method of forming the metastable liposomal and lipid nanoparticle of claim 36, wherein the apolipoprotein in the solution comprises the final concentration of between about 0.5 μg/mL to about 200 μg/mL.

38. The method of forming the metastable liposomal and lipid nanoparticle of claim 33, wherein the apolipoprotein in a solution comprises a final concentration of at least about 0.5 μg/mL.

39. (canceled)

40. The method of forming the metastable liposomal and lipid nanoparticle of claim 33, wherein the lipid component is selected from the group consisting of a dipalmitoyl phosphatidylcholine, a dimyristoyl phosphoglycerol, a palmitoyl oleoyl phosphatidylcholine, a dipalmitoyl phosphatidylcholine, a dipalmitoyl phosphatidylserine, a dipalmitoyl phosphatidylglycerol, a distearoyl phosphatidylglycerol, an egg yolk phosphatidylcholine, a soy bean phosphatidyl choline, a phosphatidylinositol, a phosphatidic acid, a sphingomyelin, a cationic phospholipid, a glycolipid and a combination thereof.

41. The method of forming the metastable liposomal and lipid nanoparticle construct of claim 33, wherein the first channel and the second channel are in a microfluidic flow cell; wherein the first fluid solution and the second fluid are in substantial laminar flow with respect to each other; and wherein a first flow rate for the first fluid solution is substantially between about 2.0 mL/min to 10.0 mL/min and wherein a second flow rate for the second fluid solution is substantially between about 1.0 mL/min to 10.0 mL/min.

42. The method of forming the metastable liposomal and lipid nanoparticle construct of claim 33, wherein a sum of a first flow rate for the first fluid solution and a second flow rate for the second fluid solution is substantially between about 2.00 mL/min to 12.00 mL/min.

43. The method of forming the metastable liposomal and lipid nanoparticle of claim 33, wherein the steps do not involve substantially shear force or cavitation.

44. The method of forming the metastable liposomal and lipid nanoparticle of claim 33, wherein the forming the metastable liposomal and lipid nanoparticle comprises forming a plurality of metastable liposomal and lipid nanoparticles that provides a substantial reproducibility between the plurality of metastable liposomal and lipid nanoparticles.

45. The method of forming the metastable liposomal and lipid nanoparticle of claim 44, wherein the substantial reproducibility between the plurality of metastable liposomal and lipid nanoparticles comprises an at least 80% batch to batch success rate.

46. The method of forming the metastable liposomal and lipid nanoparticle of claim 45 wherein the at least 80% batch to batch success rate comprises a characteristic selected from the group consisting of an at least 80% of the nanoparticles substantially not separating to any constituent parts; an at least 80% of the nanoparticles substantially maintaining a predefined particle distribution of the bioactive agent in the nanoparticle, an at least 80% of the nanoparticles substantially maintaining a predefined distribution of particle size; and a combination thereof.

47. (canceled)

48. (canceled)

49. (canceled)

50. (canceled)

51. (canceled)

52. (canceled)
